(19) United States
(12) Patent Application Publication
(10) Pub. No.: US 2005/0171510 A1
(43) Pub. Date: Aug. 4, 2005

(54) Pressure actuated safety valve with spiral flow membrane

(76) Inventors: Paul DiCarlo, Middleboro, MA (US); Karla Weaver, Framingham, MA (US)

Correspondence Address: Patrick J. Fay, Esq.;FAY KAPLUN & MARCIN, LLP, 150 Broadway, Suite 702, New York, NY, 10038, US

(21) Appl. No.: 10/768,855

(22) Filed: Jan. 29, 2004

(51) Int. Cl. A61M 25/16; A61M 5/00

(52) U.S. Cl. 604/537; 604/247

(57) ABSTRACT

A pressure actuated valve, comprises a flow chamber having an inlet and an outlet and a resilient membrane extending across the flow chamber to selectively impede passage of a fluid between the inlet and the outlet. The membrane includes at least one slit formed therein so that, when a fluid pressure to which the membrane is subjected is at least a predetermined threshold level, edges of the at least one slit separate to permit fluid flow therethrough. The at least one slit has a first curvature in a surface plane of the membrane and a second curvature along a thickness of the membrane to impart a rotational velocity component to a flow of fluid therethrough relative to a centerline of the flow chamber.

The present application incorporates by reference the entire disclosure of U.S. Application entitled “Pressure Activated Safety Valve With Anti-Adherent Coating” filed on even day herewith naming Karla Weaver and Paul DiCarlo as inventors, and U.S. Application entitled “Stacked Membrane For Pressure Actuated Valve” filed on even day herewith naming Karla Weaver and Paul DiCarlo as inventors, and U.S. Application entitled “Pressure Activated Safety Valve With High Flow Slit” filed on even day herewith naming Karla Weaver and Paul DiCarlo as inventors, and U.S. Application entitled “Dual Well Port Device” filed on even day herewith naming Katie Daly, Kristian DiMatteo and Eric Houde as inventors.

### BACKGROUND OF THE INVENTION

Many medical procedures require repeated and prolonged access to a patient's vascular system. For example, during dialysis treatment blood may be removed from the body for external filtering and purification, to make up for the inability of the patient's kidneys to carry out that function. In this process, the patient's venous blood is extracted, processed in a dialysis machine and returned to the patient. The dialysis machine purifies the blood by diffusing harmful compounds through membranes, and may add to the blood therapeutic agents, nutrients etc., as required before returning it to the patient's body. Typically the blood is extracted from a source vein (e.g., the vena cava) through a catheter sutured to the skin with a distal needle of the catheter penetrating the source vein.

It is impractical and dangerous to insert and remove the catheter for each dialysis session. Thus, the needle and catheter are generally implanted semi permanently with a distal portion of the assembly remaining within the patient in contact with the vascular system while a proximal portion of the catheter remains external to the patient's body. The proximal end is sealed after each dialysis session has been completed to prevent blood loss and infections. However, even small amounts of blood oozing into the proximal end of the catheter may be dangerous as thrombi can form therein due to coagulation. These thrombi may then be introduced into the patient's vascular system when blood flows from the dialysis machine through the catheter in a later session.

A common method of sealing the catheter after a dialysis session is to shut the catheter with a simple clamp. This method is often unsatisfactory because the repeated application of the clamp may weaken the walls of the catheter due to the stress placed on the walls at a single point. In addition, the pinched area of the catheter may not be completely sealed allowing air to enter the catheter which may coagulate any blood present within the catheter. Alternatively, valves have been used at the opening of the catheter in an attempt to prevent leaking through the catheter when the dialysis machine is disconnected. However, the unreliability of conventional valves has rendered them unsatisfactory for extended use.

### SUMMARY OF THE INVENTION

In one aspect, the present invention is directed to a pressure actuated valve, comprising a flow chamber having an inlet and an outlet and a resilient membrane extending across the flow chamber to selectively impede passage of a fluid between the inlet and the outlet, the membrane including at least one slit formed therein so that, when a fluid pressure to which the membrane is subjected is at least a predetermined threshold level, edges of the at least one slit separate to permit fluid flow therethrough, the at least one slit having a first curvature in a surface plane of the membrane and a second curvature along a thickness of the membrane to impart a rotational velocity component to a flow of fluid therethrough relative to a centerline of the flow chamber.

### BRIEF DESCRIPTION OF THE DRAWINGS

FIG. 1 shows a portion of a central line catheter according to an embodiment of the present invention;

FIG. 2 shows a cutaway view of a valve assembly including a high flow pressure activated valve membrane according to an embodiment of the present invention with the valve member in an open, in-flow configuration;

FIG. 3 shows a cutaway view of the valve assembly of FIG. 2 with the valve membrane in a closed configuration;

FIG. 4 shows a cutaway view of the valve assembly of FIG. 2 with the valve membrane in an open, out-flow configuration;

FIG. 5 shows a side view of a catheter showing a fluid stagnation region therein;

FIG. 6A shows a side view of a catheter showing a fluid stagnation region therein;

FIG. 6B shows a cross-sectional view of the catheter of FIG. 6A taken along line B-B thereof;

FIG. 7A shows a front view of a flow control membrane of a pressure actuated valve according to an embodiment of the present invention;

FIG. 7B shows a cross-sectional view of the flow control membrane of FIG. 7A taken along line A-A thereof; and

FIG. 8 is a diagram showing a top view of a flow control membrane of a pressure actuated valve including clusters of slits according to a second embodiment of the present invention.

### DETAILED DESCRIPTION

The present invention may be further understood with reference to the following description and the appended drawings, wherein like elements are referred to with the same reference numerals. The present invention is related to medical devices used to access the vascular system of a patient, and in particular to central line catheters used for chronic access to a vein or artery.

Semi-permanently placed catheters may be useful for a variety of medical procedures which require repeated access to a patient's vascular system in addition to the dialysis treatments mentioned above. For example, chemotherapy infusions may be repeated several times a week for extended periods of time. For safety reasons, as well as to improve the comfort of the patient, injections of these therapeutic agents may be better carried out with an implantable, semi-permanent vascular access catheter. Many other conditions that require chronic venous supply of therapeutic agents, nutrients, blood products or other fluids to the patient may also benefit from implantable access catheters, to avoid repeated insertion of a needle into the patient's blood vessels. Thus, although the following description focuses on dialysis, those skilled in the art will understand that the invention may be used in conjunction with any of a wide variety of procedures which require long term implantation of catheters within the body.

Examples of such implantable catheters include those manufactured by Vaxcel™, such as the Chronic Dialysis Catheter and the Implantable Vascular Access System. These devices typically are inserted under the patient's skin, and have a distal end which includes a needle used to enter a blood vessel. The devices also have a proximal end extending outside the body for connection with an outside line. These semi-permanent catheters may be sutured to the patient's skin to maintain them in place while the patient goes about his or her normal occupations.

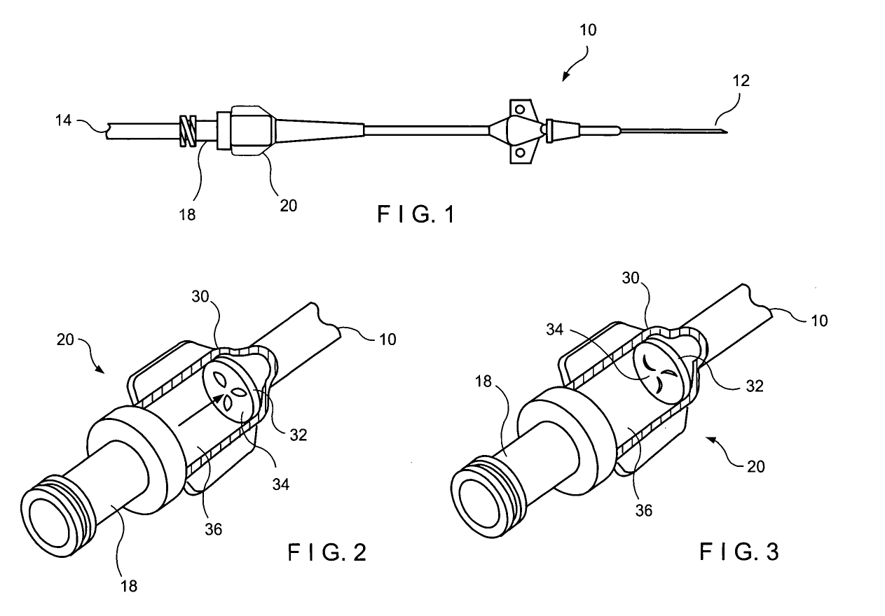

FIG. 1 shows an exemplary catheter such as, for example, the Vaxcel™ Chronic Dialysis Catheter. The catheter 10 has a distal end 12 that is insertable into a patient's vein, and which remains within the patient's body for the life of the catheter 10. The distal end 12 includes a needle (not shown) that pierces the vein of the patient to reach the flow of blood. During dialysis, blood from the patient is removed through the catheter 10, and is purified by a dialysis machine (not shown) which is connected to a hub 18 of the catheter 10 via an external line 20. The catheter 10 may include two or more lumens with a first one of the lumens being used to remove blood from the blood vessel and a second one of the lumens being used to reintroduced treated blood and/or therapeutic agents into the blood vessel. As described above, in addition to dialysis, devices similar to the catheter 10 may be used to access a patient's vascular system for other types of treatment, for example to infuse chemotherapy agents or other medications, to supply food and to remove blood samples.

When disconnected from the dialysis machine, the catheter 10 remains within the patient, connected to the patient's vascular system. Thus, it is important to securely seal the hub 18 to prevent fluids from escaping therefrom and contaminants from entering the patient's body. For example, although the proximal end 14 of the catheter 10 may be clamped to close it off, if an effective seal is not obtained, the patient runs a serious of infection as well as risks of embolisms due to air entering the blood stream and venous thrombosis due to coagulation of blood in and near the catheter. In addition, leakage from an improperly sealed catheter may expose attending medical staff to a risk of infection by blood borne pathogens. Thus a mechanism is necessary to ensure that the catheter 10 is sealed when not in use.

Conventional clamps or clips have been used to seal such catheters 10 between medical sessions. However, as the sealing forces repeatedly applied by these clips is exerted on a small portion of the surface area of the catheter 10, damage to the wall of the catheter 10 at this portion can significantly reduce the effective life of the catheter 10. It is also desired to improve the resistance of a sealing mechanism for the catheter 10 to forces applied during activities of the patient, so that the sealing mechanism will remain effective without restricting the activity of the patient. Finally, it is desired to minimize the bulk of the sealing mechanism to enhance patient comfort.

An alternative to clamping or clipping the catheter 10 is to include self sealing valves near the entrance of the flow passages of the catheter, to seal those passages when not in use. For example, the hub 18 may house one or more valve assemblies 20 which are designed to seal the lumen(s) of the catheter 10 under certain conditions, and to allow passage of fluid therethrough under other conditions. In an exemplary case applicable to a dialysis catheter, the system of valves may seal the catheter 10 when it is not connected to an operating dialysis machine, and may allow both an outflow of non-purified blood and an inflow of purified blood to the patient when an operating dialysis machine is connected thereto. These valve assemblies 20 thus selectively allow flow into or out of the patient only under predetermined conditions when they are placed in fluid contact with the inflow or outflow portions of a dialysis catheter 10.

Pressure activated safety valves (PASV's) are one type of flow control device that has been used to seal vascular catheters when not in use. These valves open when subject to flow pressure of at least a pre-determined value and remain closed when subject to pressures below the pre-determined value. In the exemplary case of a PASV used in a dialysis catheter, the valve is preferably designed so that the pre-determined pressure substantially exceeds a pressure to which the valve would be subjected from the vascular system or due to patient activity and may correspond to a pressure approximating a lower level of the pressures to which the valve would be subjected by an operating dialysis machine. Thus, when no dialysis machine is connected to the catheter, the pressure in the lumen is insufficient to open the PASV, and the catheter remains sealed.

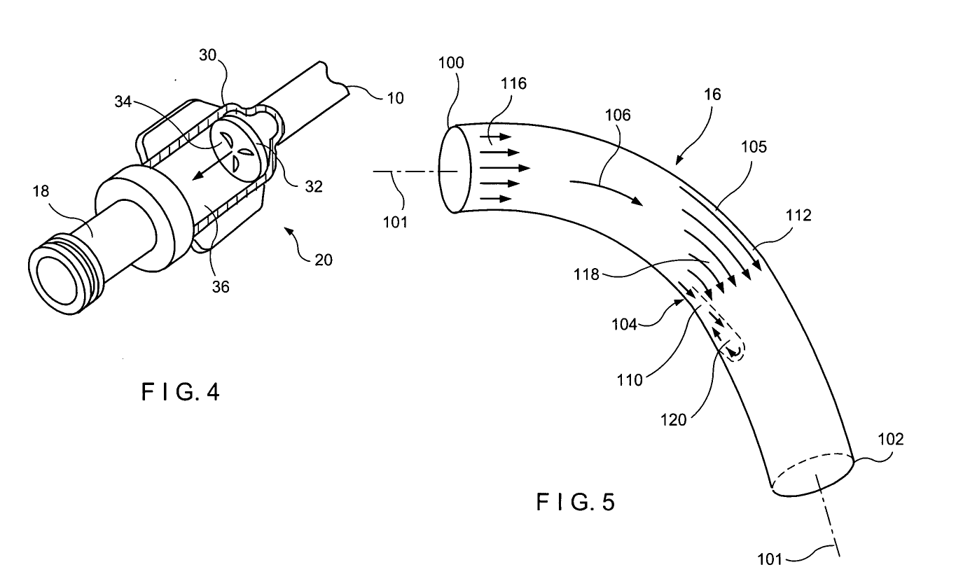

FIGS. 2-4 show more detailed views of a PASV assembly 20 in a cutaway drawing depicting three flow conditions. FIG. 2 shows a configuration of the assembly 20 in which a fluid is being introduced into catheter 10 via a hub 18 while FIG. 4 shows a configuration of the assembly 20 in which a fluid is being removed from the catheter 10 to the hub 18. FIG. 3 shows a configuration of the assembly 20 in a closed configuration in which flow therethrough is prevented. In the context of a dialysis catheter, the configurations of FIGS. 2 and 4 correspond, respectively, to blood being returned to and being withdrawn from a patient. The configuration of FIG. 3 corresponds to a condition in which no dialysis treatment is being performed, or in which a treatment has been temporarily halted so that the assembly 20 seals a lumen of the catheter 10. According to one exemplary embodiment of the present invention, the valve assembly 20 comprises a valve housing 30 forming a body of the device and a slitted membrane 32 disposed within the housing 30. The hub 18 may define the valve housing 30 or, alternatively, the housing 30 and the hub 18 may be formed as separate units. The housing 30 defines a flow chamber 36 through which fluid (e.g., blood) flows into and out of the catheter 10. The exemplary flow chamber 36 is substantially cylindrical. However in different applications, the flow chamber 36 may be of any other shape suitable for the efficient flow of a fluid therethrough.

The slitted membrane 32 may be disposed at one end of the flow chamber 36, and is positioned to selectively impede the passage of fluid though the flow chamber 36. A curved slit 34 is formed in the membrane 32 so that, only under predetermined conditions, the slit 34 is opened to permit fluid flow through the flow chamber 36. When the membrane 32 is not exposed to the predetermined conditions, the slit 34 remains closed to seal the flow chamber 36. For example, the slitted membrane 32 may be constructed so that the curved slit 34 opens only when subject to a flow pressure of at least a threshold magnitude. When a pressure to which the slitted membrane 32 is subject is less than this threshold pressure, the slit 34 remains closed. The threshold pressure may correspond, for example, to the pressure generated in the flow chamber 36 when the catheter 10 is coupled to an operating dialysis machine. In addition, the membrane 32 is preferably constructed so that the threshold pressure is significantly greater than pressures which will be generated within the catheter 10 by the vascular system or due to activities of the patient.

FIGS. 2-4 show one exemplary embodiment of a pressure activated valve assembly 20 according to the present invention. Those of skill in the art will understand that different configurations of the housing 30, the slitted membrane 32 and the slit 34 may be used without departing from the invention. For example, the membrane 32 may include one or more slits of various sizes and shapes to tailor the flow through membrane 32 and to vary the threshold pressure required to open slit 34. Those skilled in the art will understand that the shape of the membrane 32 and its placement within the housing 30 may also be varied to accommodate different designs of the housing 30.

Pressure actuated valve membranes which seal catheters when not in use have often relied on limitations in the size of the slits therethrough to ensure complete closure of the slits when not subject to at least a threshold pressure. However, this may also limit the flow rate that may be obtained through the valve membrane. Thus, it is important to ensure complete sealing of the catheter 10 while permitting an increased flow rate to allow treatment sessions to be shortened.

A pressure actuated valve constructed according to embodiments of the present invention improves the ability of a catheter attached thereto to pass a fluid at a high flow rate while retaining an effective seal when not in use. Catheters and similar devices that are inserted in the body percutaneously often undergo several compound curves along their lengths. Such a device may be tunneled subcutaneously so that its distal end may be inserted into a desired body lumen, for example, a vein or an artery. The device is then fixed to the skin of the patient to give it stability and to prevent its accidental removal. The twists and curves followed by the device may have small radii of curvature, which can result in the formation of “dead” flow areas, typically near or at the location where the tubular body of the device negotiates a sharp turn. In these regions of stagnation, the flow has a low velocity due to the inability of the fluid to follow surface boundaries of the catheter's lumen. In extreme cases, some regions may exhibit recirculation, where flow locally reverses direction. These regions of obstructed flow may form a blockage in the lumen of the device, which reduces the ability of the lumen to pass a large amount of fluid.

According to the present invention, the pressure actuated valve of the device imparts to the fluid a spiral motion with respect to a longitudinal axis of the lumen, so that the flow through the device is maximized. In particular, slits of a slitted membrane forming the flow control element of the pressure actuated valve are designed to direct the flow of fluid passing therethrough in a desired motion, and to increase the flow's turbulence. Under this desired motion, the fluid flows with a through flow velocity component as well as a rotational velocity component. The component in the through flow direction refers to movement generally between the inlet and the outlet of the valve, along the axis of the flow chamber. With respect to the catheter, the through flow direction approximately follows an axis of the catheter's lumen. The rotational velocity component is generally tangent to a circumference of the membrane and of the lumen, and causes the flow to follow a spiral or corkscrew path along the valve's flow chamber and along the lumen of the catheter.

Causing fluid to flow through the catheter 10 along a spiral path promotes a continuous exchange of fluid across various radial sectors of the flow passage cross section reducing the likelihood of stagnation of the fluid. If the shape of the flow passage causes the flow in one sector to stagnate or even reverse, the spiraling motion soon introduces higher energy (higher momentum) fluid into the low momentum sector containing the stagnating fluid. The higher momentum fluid mixes with the low momentum fluid in the stagnation regions of the curved catheter, such as the inside regions of the sharp curve, and pushes the low momentum fluid forward in the through flow direction. The result is a more uniform velocity distribution across the flow passage without the large ups and downs caused by areas of low energy stagnating flow.

FIG. 5 illustrates flow through a curved catheter body 16. In this diagram, a fluid enters the lumen of catheter body 16 through an inlet 100, and exits through an outlet 102. Inside the catheter body 16, the fluid has an average through flow velocity 106 directed generally along a centerline 101 of the catheter body 16. As can be seen, at a curve in the catheter body 16 is curved such that a portion 104 follows a small radius of curvature along an inside of the turn and a portion 105 follows a greater radius of curvature along the outside of the turn. As would be understood by those of skill in the art, fluid may enter the catheter body 16 with a velocity profile 116 having a maximum velocity near the center of the flow passage (i.e., along the centerline 101). The fluid velocity diminishes from this maximum to a minimum velocity along the walls of the flow passage due to the presence of a boundary layer caused by friction with the wall of the flow passage.

As shown in the diagram, the velocity profile changes as the fluid moves into a curved region of the catheter body 16. In the curved region, the fluid assumes a velocity distribution 118, where the largest flow velocity is found in a radial sector 112, at the outside of the curve and the lowest flow velocity occurs near the inside of the curve, in the sector 110. This effect is due in part to the greater distance the fluid has to follow along the outside curvature of the turn, but in part it is also due to the blockage to the flow that may exist in sector 110 due to a stagnation region 120. As indicated above, under certain circumstances the fluid may be unable to follow the inner curvature of the catheter body 16 in sector 110, due to the small radius of curvature of the surface that causes a separation of the boundary layer from the wall. Accordingly, the fluid in region 120 may separate from the inner wall of the catheter body 16 and form a “bubble” of fluid that is nearly stationary, or which may even move in a direction opposite to the through flow direction 106, as shown by the arrows. The stagnation bubble reduces the cross sectional area available for the fluid to flow, and thus reduces the amount of fluid that can pass through catheter body 16 in a given time.

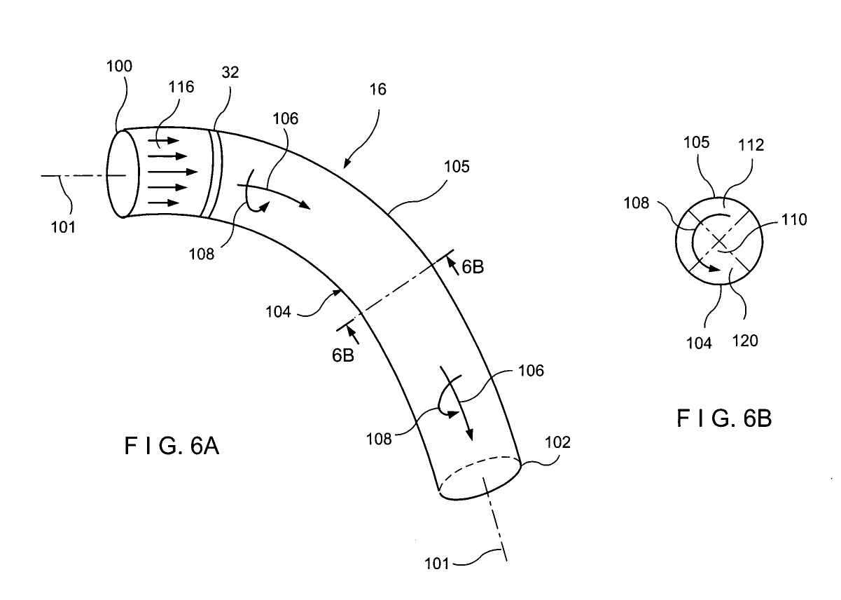

As shown in FIGS. 6A and 6B, a PASV including a slitted membrane 32 according to an exemplary embodiment of the present invention is included in a curved catheter 116 imparting a spiral flow 108 to the fluid passing therethrough to minimize the formation of stagnation regions in the catheter lumen and to maximize the flow rate through the catheter 116. As shown in FIG. 6A, flow downstream of the membrane 32 has a rotational velocity component 108, which promotes transfer of fluids between sectors of the catheter cross-section. As seen in FIG. 6B, flow moving rotationally 108 transfers fluid from sector 112 to sector 110, where the stagnation region 120 exists. The exchange of fluid from the sector 112, where the fluid moves primarily in the through flow direction, to sector 110, where the fluid stagnates, diminishes the overall amount of stagnating fluid in the lumen thereby increasing the flow rate through the catheter body 16 as a whole.

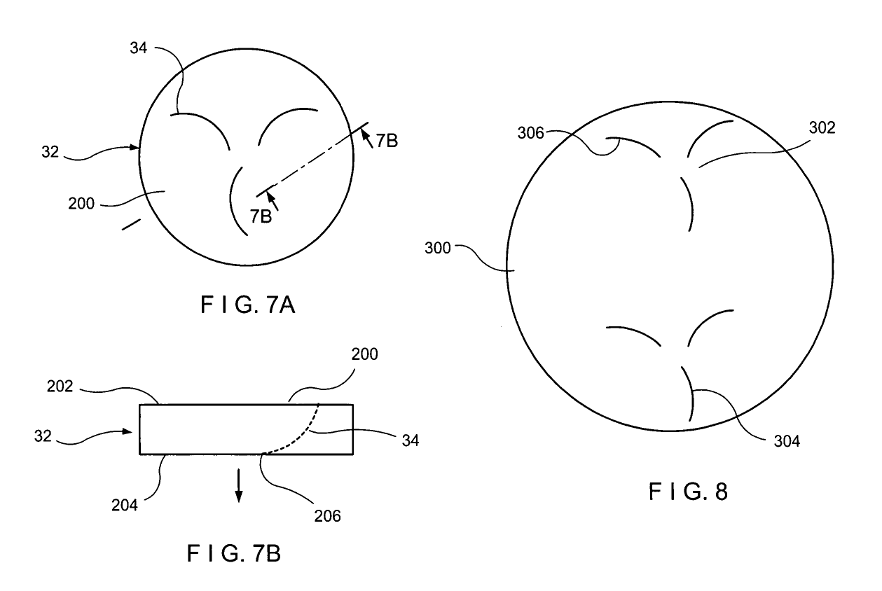

FIG. 7A shows an exemplary diagram of a flow control membrane of a slitted membrane 32 of a PASV according to the invention, which is designed to impart a spiral motion to and increase the turbulence of the fluid passing therethrough. As described above with respect to FIG. 2, the membrane 32 may be placed at an end of a flow chamber 36 of a PASV 20. According to the invention, the membrane 32 accomplishes two objectives; first it controls the flow through valve 20, so that only fluid having a pressure above a predetermined threshold can pass. Second, it imparts to the fluid the spiral flow motion that reduces the occurrence of stagnation zones in the valve 20 and in the catheter 10 downstream of the valve 20.

As shown in FIG. 7A, according to one exemplary embodiment of the present invention, the spiral flow membrane 32 comprises three curved slits 34 which are shaped to impart a rotational component to the velocity of fluid passing therethrough. Those skilled in the art will understand that the number of slits formed in the membrane 32 may be increased or decreased to obtain the desired spiral flow and overall flow volume and pressure threshold required for various applications. The slits 34 may follow a path that is defined by curves along two different planes. For example, each of the slits 34 may be curved along the surface plane of the membrane 32, as indicated in the top elevation view shown in FIG. 7A. Those skilled in the art will understand that the surface plane follows the shape of the surface 200, which, when in an unstressed state, is generally planar but which may bend and twist as fluid pressure impinges thereon. In the exemplary embodiment shown, the slits 34 are substantially arcuate along the surface plane 200 of the membrane 32, to maximize the flow surface that opens when the edges of the slits 34 separate from one another when the pressure against the membrane 32 exceeds the threshold pressure.

In addition to a curvature along the surface plane 200 of the membrane 32, the slits 34 also have a curvature in a plane extending through a thickness of the membrane 32. As shown in the cross-sectional view of FIG. 7B, this second curvature starts near an outer perimeter of the membrane 32 on an upstream face 202 thereof, and extends toward a center of a downstream face 204 of the membrane 32. This configuration of the slits 34 results in a specific pattern when the slits 34 are opened by a fluid pressure above the threshold pressure. On the upstream face 202, the slits 34 form individual openings separated radially and angularly from one another. On the downstream side 204, the slits 34 converge towards the center of the membrane 32, so that the downstream openings of the slits 34 are close to one another. Depending on the structural requirements of the membrane 32, the multiple slits 34 may converge to a single central slit located near the middle of the membrane 32, or may remain separated by small sections of solid material. The latter configuration may be used to give additional strength to the membrane 32.

A greater rotational velocity component may be imparted to the fluid by angling the slits 34 with respect to the centerline of the lumen. This angling of the slits 34 results in the fluid departing the membrane 32 at a steeper angle with respect to the centerline of the lumen thereby enhancing the swirling of the fluid. Along a surface plane 200 of the membrane 32, the slits 34 may extend along an arc to maximize a flow area of the openings created when the edges of the slits 34 are separated as the fluid pressure exceeds the threshold level, while, through the thickness of the membrane 32, the slits 34 may be curved radially and/or tangentially to enhance the spiral flow of the fluid. For example, instead of cutting the slits 34 substantially parallel to the centerline, the slits 34 may be cut so that downstream portions of the slits are radially further from or closer to the centerline than corresponding upstream portions so that the streams exiting the slits 34 swirl, for example, substantially in the manner of a braided rope. Alternatively, as would be understood by those of skill in the art, the geometry of the slits 34 may be altered in any way to impart a desired rotational component to the velocity of the fluid passing therethrough. For example, the slits 34 may be configured so that the streams of fluid emerging from the downstream face 204 of the membrane 32 merge to form a single spiralling flow through the lumen.

In a different embodiment, the upstream and/or downstream ends of the slits 34 may be connected to one another. For example, the downstream ends of two or more of the slits 34 may meet at the center of the membrane 32. In this case, the slits 34 assume an “S” shape where two slits 32 meet. The S shaped slits may potentially allow a greater amount of fluid to flow through the membrane 32. However, this configuration may reduce the structural strength of the membrane 32 relative to the membrane 32 of FIG. 7A in which the slits 34 remain separated from one another to provide a solid region of membrane material at the center to stabilize edges of the slits 34.

Fluid enters each of the slits 34 of the membrane 32 at the upstream surface 202 of the membrane 32 and is directed toward the centerline of the lumen while giving the fluid a rotational velocity component. Converging the flow towards the centerline of the catheter 116 causes the fluid to approximate the motion of blood in natural blood vessels, for example as it passes through hearth valves.

When the slits 34 converge to a single central slit 206 on downstream face 204, a predominantly unidirectional flow may be obtained. Due to the curvature of the slits 34, the membrane 32 acts as a one way flow element, favoring flow from the upstream face 202 to the downstream face 204. In the reverse direction, the flow tends to close the spiral-shaped slits 34, even if the fluid pressure exceeds the threshold pressure for the membrane 32. In fact, higher pressures simply press harder to close the spiral flow path defined by the slits 34. The pressure actuated valve 20 may therefore be optimized to act as a one way valve, by shaping the slits 34 so that flow pressure in a reverse direction pushes the slits 34 into the closed configuration.

Biological fluids such as blood generally flow through valves and other obstructions in a spiral path, often converging toward the center of the blood vessel or other passage. This flow pattern helps to minimize coagulation of the blood, and cleans solid deposits that may form in the blood vessels of other fluid passages. The embodiments of the spiral flow membrane according to the present invention mimic the natural flow patterns for biological fluids found in the body to reduce incidence of coagulation and deposition of solids in the flow passages downstream from the PASV 20 further increasing the flow rate, safety and longevity of the catheter.

According to a further embodiment shown in FIG. 8, a membrane 300 comprises multiple curved slits 306 that are grouped in clusters 302, 304. Each of the clusters 302, 304 may be tailored to produce a spiral flow as well as a spiral and converging flow downstream of the membrane 300. Combining two or more clusters 302, 304 produces a corresponding number of spiraling fluid columns, which proceed in a corkscrew motion along the walls of the catheter or other device to which the valve is connected. The fluid motion produced by the clusters 302, 304 is effective in reducing instances of stagnant flow in curved portions of the catheter is also effective in cleaning the flow path by preventing coagulation of blood or other biological fluids flowing through the catheter. In addition, such an arrangement may be useful for providing a multi lumen valve on one body, a single lumen separating into multiple lumens or multiple lumens converging into a single lumen.

It will be apparent to those skilled in the art that a different number of clusters, and a different number of slits per cluster may be used to achieve a desired flow downstream from the spiral flow membrane 300. In addition, the curved slits themselves may be modified to obtain a specific pattern of rotation of the fluid, for example by changing the pitch of the slits, the curvature, or the angle of the slits relative to the surface of the membrane. Different shapes of the spiral flow of fluid downstream of the membrane may be obtained, to suit specific devices and to maximize the flow through the lumen of a specific medical device under given flow conditions.

In an exemplary embodiment of the pressure actuated valve according to the invention, the spiral flow membrane is formed of a polymeric material, for example silicone or latex. A variety of other flexible materials may be used, however, for the same purpose. The complex shapes of the curved slits and the clusters of slits may be obtained by injection molding methods, or by stamping polymeric sheets. It will be apparent to those skilled in the art that conventional methods may be used to obtain the desired shape and configuration of the spiral flow membranes described herein.

The present invention has been described with reference to specific embodiments, and more specifically to a spiral flow membrane used in a pressure actuated safety valve attached to a catheter. However, other embodiments may be devised that are applicable to other medical devices, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

1. A pressure actuated valve, comprising:
a flow chamber having an inlet and an outlet; and
a resilient membrane extending across the flow chamber to selectively impede passage of a fluid between the inlet and the outlet, the membrane including at least one slit formed therein so that, when a fluid pressure to which the membrane is subjected is at least a predetermined threshold level, edges of the at least one slit separate to permit fluid flow therethrough, the at least one slit having a first curvature in a surface plane of the membrane and a second curvature along a thickness of the membrane to impart a rotational velocity component to a flow of fluid therethrough relative to a centerline of the flow chamber.

2. The valve according to claim 1, wherein, when the fluid pressure to which the membrane is subjected is less than the threshold level, the membrane biases the edges of the at least one slit to remain in contact with one another to prevent fluid flow therethrough.

3. The valve according to claim 1, wherein the at least one slit includes a plurality of slits.

4. The valve according to claim 1, wherein the second curvature comprises a radial curvature component and a tangential curvature component.

5. The valve according to claim 3, wherein the slits are disposed substantially symmetrically with respect to a center of the membrane.

6. The valve according to claim 3, wherein the slits are disposed substantially symmetrically with respect to a through-flow direction of the fluid.

7. The valve according to claim 1, wherein the first curvature is substantially arcuate.

8. The valve according to claim 1, wherein the second curvature extends substantially radially from a peripheral portion of the membrane toward a center thereof.

9. The valve according to claim 3, wherein the membrane defines a first face facing the inlet and a second face facing the outlet and wherein the slits intersect on one of the first and second faces.

10. The valve according to claim 1, wherein the first and second curvatures are selected to direct fluid flow through the at least one slit at a selected angle relative to a centerline of the flow chamber.

11. The valve according to claim 3, wherein the slits cooperate to impart the rotational velocity component to fluid flow therethrough.

12. The valve according to claim 3, wherein the slits cooperate to impart turbulence to fluid flow therethrough.

13. The valve according to claim 3, wherein the slits are grouped in clusters, the clusters being disposed substantially symmetrically about a center of the membrane.

14. The valve according to claim 13, wherein each of the clusters comprises three of the slits.

15. A catheter comprising:
a distal end adapted for insertion into a body lumen;
a proximal end adapted for connection to a medical device;
a flow channel extending between the distal and proximal ends and adapted to convey a fluid therebetween; and
a membrane extending across the flow channel to selectively impede fluid flow therethrough, the membrane including at least one slit extending therethrough so that, when a fluid pressure to which the membrane is subjected is at least a predetermined threshold level, edges of the at least one slit separate to permit fluid flow therethrough and, when the fluid pressure to which the membrane is subjected is less than the threshold level, the edges of the at least one slit remain in contact with one another to prevent fluid flow therethrough, the at least one slit following a curved plane adapted to impart a velocity component to the fluid passing therethrough rotational with respect to a centerline of the flow channel.

16. The catheter according to claim 15, wherein the at least one slit includes a plurality of slits.

17. The catheter according to claim 16, wherein the slits are arranged to cooperate to impart a substantially helical motion to fluid flowing through the flow channel.

18. The catheter according to claim 17, wherein the slits are arranged in clusters of slits, the clusters being arranged substantially symmetrically about a point of the membrane.

19. A pressure actuated valve, comprising:
a flow channel extending between an inlet and an outlet of the valve; and
a resilient membrane extending across the flow channel, the membrane including a plurality of slits extending therethrough, each of the slits curving with respect to a centerline of the flow channel and with respect to a plane substantially perpendicular to the centerline, wherein the membrane biases the slits to a closed configuration in which edges of the slits contact one another to seal the flow channel when a pressure to which the membrane is subjected is less than a predetermined threshold level and wherein, when the pressure to which the membrane is subjected exceeds the threshold level, the edges of the slits separate from one another to permit flow therethrough, the curvature of the slits imparting a component to the velocity of flow therethough that is rotational with respect to the centerline.

20. The valve according to claim 19, wherein the curvature of the slits with respect to the centerline comprises a radial curvature component and a tangential curvature component.

21. The valve according to claim 19, wherein the curvature of the slits with respect to a plane substantially perpendicular to the centerline is substantially arcuate.

22. The valve according to claim 19, wherein at least two of the slits converge on one face of the membrane.

23. The valve according to claim 19, wherein the slits are arranged in a plurality of clusters, the clusters being disposed substantially symmetrically on the membrane.

24. The valve according to claim 23, wherein the clusters are disposed substantially symmetrically with respect to a center of the membrane.

25. The valve according to claim 19, wherein the membrane is formed of a polymeric material.

26. The valve according to claim 19, wherein the membrane is formed of one of silicone and latex.

27. The valve according to claim 19, wherein the path of each of the plurality of slits is selected to provide a spiral flow of fluid downstream from the valve.
